(19) United States
(12) Patent Application Publication
(10) Pub. No.: US 2023/0368908 A1
(43) Pub. Date: Nov. 16, 2023

(54) ESTIMATING RESIDUAL RENAL FUNCTION

(71) Applicant: GAMBRO LUNDIA AB, Lund (SE)

(72) Inventor: Jan STERNBY, Lund (SE)

(21) Appl. No.: 18/030,116

(22) PCT Filed: Sep. 27, 2021
(86) PCT No.: PCT/EP2021/076441

§ 371 Date: Apr. 4, 2023

(30) Foreign Application Priority Data
Oct. 5, 2020 (SE) 2051169-7

(51) Int. Cl. G16H 40/63; G16H 10/40; G16H 50/70; G16H 50/30

(52) U.S. Cl. CPC G16H 40/63; G16H 10/40; G16H 50/70; G16H 50/30; A61B 10/007

(57) ABSTRACT

A computer system is configured to implement a method of estimating residual renal function in a dialysis patient based on data for two consecutive treatment sessions of intermittent dialysis therapy. The computer system obtains first and second blood concentration values (C1, C2) for a substance, e.g. urea, creatinine, at a start and an end of a first treatment session, and a third blood concentration value (C3) at a start of a second treatment session. The computer system also obtains time points (t1, t2, t3) for the start and the end of the first treatment session and the start of the second treatment session. The computer system then estimates the residual renal function as a function of the first, second, and third concentration values (C1, C2, C3), and the time points (t1, t2, t3). The method obviates the need to include data representing collection of urine from the patient.

### TECHNICAL FIELD

The present disclosure relates to techniques for estimating the residual renal function in dialysis patients.

### BACKGROUND ART

Patients with kidney failure are treated by dialysis therapy, such as hemodialysis and peritoneal dialysis. Even if dialysis therapy is an effective treatment, it does not replace all aspects of the function of the native kidneys. The ability of the native kidneys to eliminate water and uremic toxins in patients with kidney failure is commonly referred to as the residual renal function (RRF) or the residual kidney function (RKF). Any residual renal function is considered valuable to the dialysis patient. RRF is considered in the prescription of both hemodialysis and peritoneal dialysis and interventions to preserve RRF are an active area of clinical research. The caretaker thus has an interest in quantifying the residual renal function.

Often, the glomerular filtration rate (GFR) is used as a measure of RRF and describes the flow rate of filtered fluid through the kidney. Conventional techniques for estimating GFR rely on 24 hour collection of urine, which is quite cumbersome and error prone, for example caused by errors in collection and incomplete bladder emptying. The determination of residual clearance involves measuring the concentration of a marker in the collected urine and multiplying by the total urine volume to get the total marker removal, which is divided by the mean serum concentration of the marker during the collection period. The mean serum concentration may be obtained as the mean of measured serum concentrations at the beginning and end of the collection period. The marker may be clearance of an exogenously administered marker, such as inulin, or an endogenous marker, such as urea or creatinine. Residual urea clearance determined in this way will underestimate GFR, since some of the urea in the primary urine is reabsorbed in the renal tubules. On the other hand, creatinine is secreted directly into the renal tubules, so that residual creatinine clearance will overestimate the GFR. Therefore, GFR is often estimated as a mean value of the residual urea clearance and the residual creatinine clearance.

### SUMMARY

It is an objective to at least partly overcome one or more limitations of the prior art.

A further objective is to provide an alternative technique for estimating the residual renal function in a dialysis patient.

Another objective is to provide such a technique for which measurement errors are less of a problem and which is simple to perform.

One or more of these objectives, as well as further objectives that may appear from the description below, are at least partly achieved by a computer-implemented method, a computer-readable medium and a computer device in accordance with the independent claims, embodiments thereof being defined by the dependent claims.

A first aspect is a computer-implemented method of estimating a residual renal function, RRF, in a dialysis patient. The method comprises: obtaining first and second concentration values for a substance in the blood of the dialysis patient at start and end of a first treatment session of intermittent dialysis therapy, and a third concentration value for a substance in the blood of the dialysis patient at start of a second treatment session, which is consecutive to the first treatment session; obtaining time points for the start and the end of the first treatment session and the start of the second treatment session; and calculating an estimation value of the RRF as a function of the first, second and third concentration values, and the time points.

The change in concentration of a substance in the blood of the dialysis patient during a treatment session is mainly attributed to the effect of the dialysis therapy. The change in concentration of the substance in the blood of the dialysis patient between treatment sessions will mainly be caused by generation of the substance in the dialysis patient. The first aspect is based on the insight that the change in concentration during the treatment session will also be affected by the generation of the substance in the patient and by the clearance of the substance by RRF (if present), and that the change in concentration between treatment sessions will also be influenced by RRF. There is thus an interdependence, albeit complex, between the change in concentration, the generation rate in the patient, and the RRF. Ignoring the complexity, this means that it is possible to use measurement data obtained over a time period with on-going dialysis therapy to estimate both the unknown RRF and the unknown generation rate of the substance in the patient. By further insightful reasoning, it has been found that measurement data including three well-timed concentration values of the substance in the blood of the dialysis patient spanning a treatment session and time period between the treatment session and the next treatment session, together with treatment data readily available for all intermittent dialysis therapies, will provide enough input data to estimate both RRF and generation rate.

The first aspect provides a novel and alternative technique for estimating RRF in a dialysis patient. The first aspect may be implemented for any intermittent dialysis therapy and any combinations of such therapies. The first aspect provides a technique which is based on measurement data that may be acquired by simple and straight-forward blood sampling and analysis procedures. For example, blood sample measurements of urea and creatinine are regularly performed at the start and end of a treatment session to calculate the dialysis adequacy. Thus, it is possible to perform measurements by use of commercially available and standard equipment to obtain the concentration values for use by the first aspect. This will result in a cost-effective and time-efficient procedure. It may particularly be noted that the technique does not require, and thus is independent of, any data representing urine collected from the dialysis patient. The first aspect thereby obviates the sources of error associated with urine collection.

Some embodiments of the first aspect are based on the further insight that the generation rate of the substance may be derived by simple and straight-forward calculation by use of a parameter known as “standard Kt/V” in the art, commonly abbreviated “stdKt/V”. This parameter is a well-known and established measure of dialysis adequacy and has been developed to enable comparison of a broad spectrum of dialysis therapies, including intermittent hemodialysis therapies, continuous and intermittent ultrafiltration therapies, continuous and intermittent peritoneal dialysis, and continuous hemodialysis therapies for acute renal failure. Although the parameter is commonly derived for urea, it is generally applicable to any substance that is extracted from the blood of the dialysis patient in dialysis therapy. In accordance with its underlying definition, stdKt/V is given as G·T/(C̅s̅·V), where G is the generation rate of a substance in the dialysis patient, T is a predefined time period, C̅s̅ is the average pre-dialysis concentration of the substance in the blood of the dialysis patient over the time period T, and V is the distribution volume in the dialysis patient of the substance. By insightful reasoning, it has been realized that if stdKt/V is known, it is possible to apply the underlying definition of stdKt/V to directly compute the generation rate G or the relative generation rate G/V based on the known value of stdKt/V, provided that the average pre-dialysis concentration C̅s̅ is measured or estimated for the time period T.

A number of different computation algorithms have been developed that relate stdKt/V to known or measurable parameters of dialysis therapy. The computation algorithms are based on the assumption that the concentration of the substance in the blood of the dialysis patient is substantially equal at the start and end of the time period T. In other words, the computation algorithms for stdKt/V assume that the dialysis patient attains a steady state in concentration profile over the time period T. Generally, the existing computation algorithms for stdKt/V are given either as a function of the session Kt/V of the substance for the respective treatment session within the time period T, or as a function of the blood concentrations of the substance at the start and end of the respective treatment session within the time period T. The computation algorithms for stdKt/V further operate on the duration of the respective treatment session and the total fluid volume (if any) removed from the blood by dialysis therapy over the time period T. Any such computation algorithms that also account for RRF may be used in embodiments of the first aspect.

In the following, various embodiments of the first aspect are defined. These embodiments provide at least some of the technical effects and advantages described in the foregoing, as well as additional technical effects and advantages as readily understood by the skilled person, e.g. in view of the following detailed description.

In some embodiments, the first, second and third concentration values represent equilibrated concentrations of the substance in the blood of the dialysis patient.

In some embodiments, the first and second treatment sessions are performed during a therapy time period, and said calculating the estimation value is based on an assumption that the concentration of the substance in the blood of the dialysis patient is substantially equal at the start and end of the therapy time period.

In some embodiments, the therapy time period comprises a week.

In some embodiments, said calculating the estimation value comprises: representing an unknown generation rate of the substance in the dialysis patient by a functional dependence on standard Kt/V for the therapy time period, and representing the standard Kt/V by a predefined estimation function, which is based on said assumption and operates on the time points, the therapy time period, an unknown session Kt/V of the first treatment session, and the RRF to be estimated.

In some embodiments, said functional dependence comprises multiplying the standard Kt/V, and an estimated concentration value, which is representative of an average pre-dialysis concentration of the substance in the blood of the dialysis patient during the therapy time period, and the reciprocal of the therapy time period.

In some embodiments, the method further comprises: calculating the estimated concentration value as a function of at least one of the first and third concentration values.

In some embodiments, the method further comprises: calculating the estimated concentration value as a weighted average of at least the first and third concentration values.

In some embodiments, said calculating the estimation value comprises: generating a plurality of data points, each of the data points comprising a unique combination of candidate values of the session Kt/V and the RRF; calculating, for a respective data point, an associated value of the generation rate by use of the predefined estimation function populated by the time points; finding a matching data point among the plurality of data points; and setting the estimation value to the candidate value of the RRF in the matching data point; wherein the candidate values of the matching data point match an apparent session Kt/V for the first treatment session and an apparent RRF, wherein the apparent session Kt/V is a function of the first and second concentration values, the time points for the start and the end of the first treatment session, and the associated value of the generation rate for the matching data point, and wherein the apparent RRF is a function of the second and third concentration values, the time points for the end of the first treatment session and the start of the second treatment session, the associated value of the generation rate for the matching data point.

In some embodiments, said finding comprises: operating intermediate functions on the respective data point and the associated value of the generation rate, to calculate first, second, third and fourth intermediate values; operating scaling functions on the respective data point, to calculate first and second scale factors; and determining the estimation value as a function of the plurality of data points, the first, second, third and fourth intermediate values generated for the respective data point, and the first and second scale factors second generated for the respective data point.

In some embodiments, the intermediate functions are populated by the first, second and third concentration values, and the time points, and the scaling functions are populated by the time points for the end of the first treatment session and the start of the second treatment session.

In some embodiments, the intermediate functions are given by:

[symbol]

=C1−G/V·(t2−t1)/(eKt/V−β)

[symbol]

=C2−G/V·(t2−t1)/(eKt/V−β)

[symbol]

=C2−G/V·(t3−t2)/(Kr/V·(t3−t2)+β)

[symbol]

=C3−G/V·(t3−t2)/(Kr/V·(t3−t2)+β)

with [symbol] , [symbol] , [symbol] , [symbol] being the first, second, third and fourth intermediate values, C1, C2, C3 being the first, second and third concentration values, t1, t2 being the time points at the start and the end of the first treatment session, t3 being the time point at the start of the second treatment session, G/V being the associated value of the generation rate, eKt/V being the session Kt/V of the respective data point, Kr/V representing the RRF, β being a ratio of total fluid volume removed from the blood during the first treatment session and distribution volume in the dialysis patient, wherein the scaling functions are given by:

M=exp(−ln(1+β)/β·eKt/V+ln(1+β))

N=exp(−ln(1+β)/β·Kr/V·(t3−t2)−ln(1+β))

with M, N being the first and second scale factors.

In some embodiments, said determining the estimation value comprises: finding, among the plurality of data points, a fitting data point for which the second intermediate value substantially equals the first intermediate value multiplied by the first scaling factor, and for which the fourth intermediate value substantially equals the third intermediate value multiplied by the second scale factor, wherein the matching data point is determined based on the fitting data point.

In some embodiments, the plurality of data points defines a first grid of unique combinations of candidate values, said method further comprising: generating a second plurality of data points that defines a second grid of unique combinations of candidate values, the second grid being smaller and having a higher resolution than the first grid and being located around the fitting data point; repeating said calculating the estimation value for the second plurality of data points; and finding, among the plurality of data points, a second fitting data point, wherein the second fitting data point forms the matching data point.

In some embodiments, said calculating the estimation value comprises: setting a first candidate value of the RRF and a second candidate value of the session Kt/V; calculating a reference value of the generation rate by use of the predefined estimation function populated by the time points, the therapy time period, and the first and second candidate values; calculating a comparison score indicative of the first candidate value in relation to an apparent RRF given as a function of the reference value, the second and third values, and the time points for the end of the first treatment session and the start of the second treatment session; iteratively modifying the first candidate value while calculating the reference value and the comparison score until the comparison score fulfils a convergence criterion; and setting the estimation value to the candidate value for which the comparison score fulfils the convergence criterion.

In some embodiments, said setting the second candidate value comprises: operating an approximation function on the first and second concentration values to calculate an estimated session Kt/V, and setting the second candidate value based on the estimated session Kt/V.

In some embodiments, said calculating the reference value comprises: calculating the standard Kt/V by operating the predefined estimation function on the time points, the therapy time period, and the first and second candidate values; calculating an estimated generation rate based on the standard Kt/V; and determining the reference value based on the estimated generation rate.

In some embodiments, said calculating the reference value further comprises: updating the second candidate value by operating a refinement function on the estimated generation rate, the first and second concentration values, and the time points for the start and end of the first treatment session; and repeatedly performing said calculating the standard Kt/V, said calculating the estimated generation rate and said updating the second candidate value, until a second convergence criterion is fulfilled, wherein said determining sets the reference value to the estimated generation rate for which the second convergence criterion is fulfilled.

In some embodiments, said calculating the comparison score comprises: operating intermediate functions on the reference value and the first candidate value, to calculate first and second intermediate values; and operating a scaling function on the first candidate value, to calculate a scale factor, wherein the comparison score defines a relation between the second intermediate value to the product of the first intermediate value and the scale factor.

In some embodiments, the intermediate functions are populated by the second and third concentration values, and the time points for the end of the first treatment session and the start of the second treatment session, and the scaling function is populated by the time points for the end of the first treatment session and the start of the second treatment session.

In some embodiments, the comparison score is calculated to be further indicative of the second candidate value in relation to an apparent session Kt/V given as a function of the reference value, the first and second values, and the time points for the start and the end of the first treatment session; and the first and second candidate values are iteratively modified until the comparison score fulfils the convergence criterion.

In some embodiments, the method further comprises one or more of: displaying the estimation value, evaluating the estimation value for assessment of a physiological status of the dialysis patient, and displaying an indicator of the physiological status of the dialysis patient.

In some embodiments, said substance is one of urea, creatinine, beta-2-microglobin, β-trace protein, or cystatin C.

In some embodiments, the method further comprises: obtaining a volume value representative of total fluid volume removed from the blood during the first treatment session, and wherein the estimation value of the RRF is further calculated as a function of volume value.

A second aspect is a computer-readable medium comprising computer instructions which, when executed by a processor, cause the processor to perform the method of the first aspect or any of its embodiments.

A third aspect is a computer system for estimating a residual renal function, RRF, in a dialysis patient. The computer system is configured to perform the method of the first aspect or any of its embodiments.

Still other objectives, features, embodiments, aspects and technical effects may appear from the following detailed description, from the attached claims as well as from the drawings.

### BRIEF DESCRIPTION OF THE DRAWINGS

Embodiments will now be described in more detail with reference to the accompanying drawings.

FIG. 1 is a schematic overview of a system for estimation of the RRF of a dialysis patient.

FIG. 2 is an example graph of equilibrated blood urea concentration of a dialysis patient that undergoes three dialysis treatment sessions during the course of a week.

FIG. 3A is a flow chart of an example method of estimating the RRF of a dialysis patient, and FIG. 3B-3C are flow charts of example calculation procedures in the method of FIG. 3A in accordance with a first approach.

FIG. 4 is a block diagram of functional blocks, and associated input and output data, of an example computer system for performing the method of FIG. 3C.

FIG. 5A is a graph of contour curves calculated for determination of an estimation value of the RRF, and FIG. 5B illustrates an example of first and second grids of candidate data points used for determining the estimation value in accordance with an embodiment.

FIGS. 6A-6C are flow charts of example calculation procedures in the method of FIG. 3A in accordance with a second approach.

FIG. 7 is a block diagram of functional blocks, and associated input and output data, of an example computer system for performing the procedures in FIGS. 6A-6B.

### DETAILED DESCRIPTION OF EXAMPLE EMBODIMENTS

Embodiments will now be described more fully hereinafter with reference to the accompanying drawings, in which some, but not all, embodiments are shown. Indeed, the subject of the present disclosure may be embodied in many different forms and should not be construed as limited to the embodiments set forth herein; rather, these embodiments are provided so that this disclosure may satisfy applicable legal requirements. Like numbers refer to like elements throughout.

Also, it will be understood that, where possible, any of the advantages, features, functions, devices, and/or operational aspects of any of the embodiments described and/or contemplated herein may be included in any of the other embodiments described and/or contemplated herein, and/or vice versa. In addition, where possible, any terms expressed in the singular form herein are meant to also include the plural form and/or vice versa, unless explicitly stated otherwise. As used herein, “at least one” shall mean “one or more” and these phrases are intended to be interchangeable. Accordingly, the terms “a” and/or “an” shall mean “at least one” or “one or more”, even though the phrase “one or more” or “at least one” is also used herein. As used herein, except where the context requires otherwise owing to express language or necessary implication, the word “comprise” or variations such as “comprises” or “comprising” is used in an inclusive sense, that is, to specify the presence of the stated features but not to preclude the presence or addition of further features in various embodiments. Similarly, the expressions “as a function of” and “based on” in combination with a specified set of parameters or the like are inclusive and do not to preclude the presence or addition of further parameters.

The following description refers to “standard Kt/V”, also known as standardized Kt/V or stdKt/V, which is an established measure of dialysis adequacy. The underlying motivation for developing this measure was a need be able to compare the dialysis doses provided by different types of dialysis therapies and combinations of dialysis therapies, including both continuous and intermittent therapies. The measure was first presented by Frank Gotch in the article “The current place of urea kinetic modelling with respect to difference dialysis modalities”, published in Nephrol Dial Transplant. 13 [Suppl 6]: 10-14 (1998), which is incorporated herein by reference. Essentially, Gotch presented a method of downgrading intermittent dialyzer clearances to the equivalent of a continuous clearance (stdK) by redefining clearance as the urea generation rate divided by the average pre-dialysis blood urea concentration. The definition assumes that the blood urea concentration is the same at the start and end of the time period t. Specifically, Gotch provided the following definition of stdKt/V:

$$stdKt / V = \frac{G \cdot T}{\overline{Cs} \cdot V}$$

where G is the average generation rate of urea in the dialysis patient over a predefined time period T, C̅s̅ is the average of the blood urea concentrations at the onset of the treatment sessions that are performed during the time period T, and V is the distribution volume in the patient. The time period T may include any number of treatment sessions. Traditionally, the time period T is set to a week, resulting in a “weekly stdKt/V”, but any other time period may be used, e.g. a day, 2 weeks, a month, etc. The time period T is an “equalization period” since the dialysis therapy is assumed to bring the patient's blood urea concentration back to the start value after the time period. In the following, the time period T is denoted “therapy time period” or “therapy period”. Although Gotch gave the definition for urea, it is equally applicable to any other substance that is exchanged with the blood of a dialysis patient during dialysis therapy.

Over time, stdKt/V has become an established measure and is included in KDOQI—Kidney Disease Outcomes Quality Initiative, which is a broadly accepted clinical practice guideline in nephrology, see “KDOQI Clinical Practice Guideline for Hemodialysis Adequacy: 2015 Update”, Am J Kidney Dis. 2015;66(5), pages 908-912: “Guideline 3: Measurement of Dialysis—Urea Kinetics”. The rationale behind and established use of stdKt/V is also discussed in the reference book “Replacement of Renal Function by Dialysis”, 5th revised edition, 2004, editors Hörl, Koch, Lindsay, Ronco and Winchester, Chapter 22—Adequacy of hemodialysis, pages 597-638, as well as in the article “Assessing the Adequacy of Small Solute Clearance for Various Dialysis Modalities, with Inclusion of Residual Native Kidney Function”, by Chin et al, published in Seminars in Dialysis, 30(3), 235-240 (2017).

It is important to understand that stdKt/V is not the same as Kt/V, which is an established measure that describes the effect (“dialysis dose”) of a single treatment session and is theoretically given by the logarithm of the ratio of the pre- and post-dialysis urea concentrations. More specialized equations have been developed to account for urea generation and ultrafiltration, e.g. so-called single-pool Kt/V (spKt/V), and to also account for the distribution of urea in the patient, e.g. so-called equilibrated Kt/V (eKt/V). In the following, the Kt/V for a single treatment session is denoted “session Kt/V” to be distinguished from stdKt/V.

In a clinical situation, it is difficult to calculate stdKt/V based on its definition given that at least G is unknown. Therefore, various algorithms for computing or estimating stdKt/V have been developed. One computation algorithm is proposed by Daugirdas et al. in the article “Standard Kt/Vurea: a method of calculation that includes effects of fluid removal and residual kidney clearance”, published in Kidney Int 77: 637-644 (2010). This algorithm accounts for UF (ultrafiltration) and RRF and works well if treatment sessions are equal and evenly distributed over the week. A further computation algorithm is proposed by Sternby in the article “Mathematical Representation of Standard Kt/V Including Ultrafiltration and Residual Renal Function”, published in ASAIO J. 64(5), e88-e93 (2018). This algorithm enables calculation of stdKt/V irrespective the nature, number and spacing of treatment sessions and accounts for both UF and RRF. Both of the foregoing publications are incorporated herein in their entirety by this reference.

Common to these computation algorithms is that they enable calculation of stdKt/V from input data that includes either pre- and post-dialysis urea concentrations in the patient's blood for one or more treatment sessions during the therapy period T, or the session Kt/V of urea for the one or more treatment sessions, as well as the start and end time points for the respective session, the total ultrafiltration volume (UFV) removed from the blood during the during the therapy period T, and the residual renal function (RRF) of the patient.

In the foregoing, a distinction is made between intermittent and continuous dialysis therapies. As used herein, “continuous dialysis therapy” refers to any renal replacement therapy that is operated continuously on the patient over the therapy period T, such that the concentration of urea (or another substance) remains essentially constant in the blood of the patient. In contrast, “intermittent dialysis therapy” involves one or more renal replacement therapies each of which is operated on the patient during a respective subset of the therapy period T, causing the concentration of urea (or another substance) to vary during the therapy period T. Such renal replacement therapies may include one or more of hemodialysis, hemodiafiltration, hemofiltration, ultrafiltration and peritoneal dialysis.

The disclosure may refer to an “equilibrated” concentration of a substance in blood. This is the concentration of the substance in the blood when the body is in equilibrium and is thus equal to the average concentration in the distribution volume within the body of an individual. The blood concentration measured at the start of a session (“pre-dialysis concentration”) will inherently be an equilibrated value. However for many substances, the blood concentration measured at the end of a session (“post-dialysis concentration”) will differ from the equilibrated concentration due to recirculation and rebound effects. There are a number of established methods for deriving an equilibrated value for the post-dialysis concentration. One method is to measure the blood concentration at a predefined time before the end of the session. For urea, this time period is typically 30-40 minutes. Another method is to convert the measured blood concentration at the end of the treatment session into an equilibrated value by use of any available conversion algorithm.

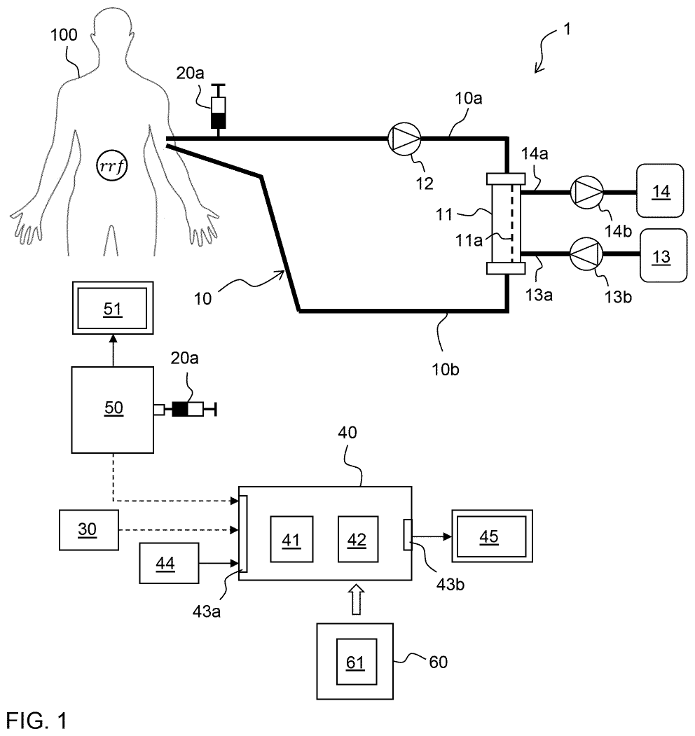

Embodiments will now be exemplified for measurements of urea and calculation of residual clearance, designated by Kr, as an estimation value of RRF. Reference is made to FIG. 1, which schematically depicts a blood treatment system 1 for performing hemodialysis when connected to a patient 100, i.e. a human subject. It is assumed that the patient 100 has a residual renal function, as indicated by rrf in FIG. 1.

The system 1 comprises an extracorporeal blood circuit (“EC circuit”) 10 which is connected to the vascular system of the patient 100 at a blood withdrawal end and a blood return end. The connections may be performed by any conventional device, such as a needle or catheter. Blood lines or tubings are arranged to define a blood withdrawal path or limb 10a and a blood return path or limb 10b of the EC circuit 10. A blood filtration unit 11, denoted “dialyzer” herein, is connected between the withdrawal and return paths 10a, 10b. The dialyzer 11 comprises a semi-permeable membrane 11a, which is arranged to separate the dialyzer 11 into a blood compartment, which is fluidly connected to the withdrawal and return paths 10a, 10b, and a dialysis fluid compartment. A blood pump 12 is arranged in the withdrawal path 10a and is operable to draw blood from the patient 100 and pump the blood via the blood compartment of the dialyzer 11 and through the return path 10b back to the patient 100. The system 1 further comprises a source 13 of dialysis fluid. A dialysis fluid path or line 13a connects the source 13 to the dialysis fluid compartment of the dialyzer 11. Similarly, an effluent path or line 14a connects the dialysis fluid compartment of the dialyzer 11 to a sink 14 for spent dialysis fluid (also known as “effluent”). A dialysis fluid pump 13b is arranged in the dialysis fluid path 13a, and an effluent pump 14b is arranged in the effluent path 14a. The skilled person understands that the blood treatment system 1 may include further components, such as a venous drip chamber, clamps, sensors, etc.

A control device 30 is configured to generate control signals for operative components of the system 1, such as the pumps 12, 13b, 14b, to cause the system 1 to perform a treatment session in accordance with settings that have been entered into the control device 30, e.g. by a caretaker or the patient 100. The operation of a hemodialysis system 1 is known to the person skilled in the art and will not be detailed here.

FIG. 1 also illustrates a sampling device 20a, which is used for taking a sample of the patient's blood, either from the withdrawal path 10a of the EC circuit 10 (as shown) or directly from the vascular system in the patient 100. As shown, the sampling device 20a may then be connected to a blood analysis apparatus 50, which is separate from the system 1 and configured to analyze the blood sample for determination of the concentration of one or more substances, including urea. The apparatus 50 may present the result of the analysis of the respective blood sample to the operator on a display device 51. Alternatively, the blood sample may be subjected to manual laboratory analysis to yield the concentration. As known in the art, the urea concentration in blood may be given in terms of the entire urea molecule or its nitrogen-content (commonly denoted “blood urea nitrogen”, BUN).

FIG. 1 also depicts a computation device or computer system 40 which is configured to perform dedicated calculations to generate output data that allows a clinician to assess the RRF of the patient 100. The computer system 40, which may or may not be part of a dialysis machine, comprises a processor 41 and computer memory 42. A computer program is stored in the memory 42 and executed by the processor 41 to perform the calculations. As indicated, the computer program 61 may be supplied to the computer system 40 on a computer-readable medium 60, which may be a tangible (non-transitory) product (e.g. magnetic medium, optical disk, read-only memory, flash memory, etc.) or a propagating signal. In the illustrated example, the computer system 40 comprises an input interface 43a for connection to one or more input devices 44 that enable an operator to supply input data, as well as an output interface 43b for connection to one or more output devices 45 for providing output data to the operator. For example, the input device(s) 44 may comprise a keyboard, keypad, computer mouse, control button, touch screen, a microphone, etc., and the output device(s) 45 may comprise a display device, an indicator lamp, an alarm device, a speaker, a printer, etc.

The operator may enter input data, e.g. including the blood concentration values, into the computer system 40 via the input device 44. Alternatively or additionally, as indicated by a dashed arrow in FIG. 1, the blood analysis apparatus 50 may be connected, by wire or wirelessly, to the input interface 43a to transfer the blood concentration values to the computer system 40. Alternatively or additionally, as indicated by a dashed arrow in FIG. 1, the control device 30 may be similarly connected to transfer input data to the computer system 40. It is also conceivable that the computer system 40 is integrated in the control device 30, or vice versa.

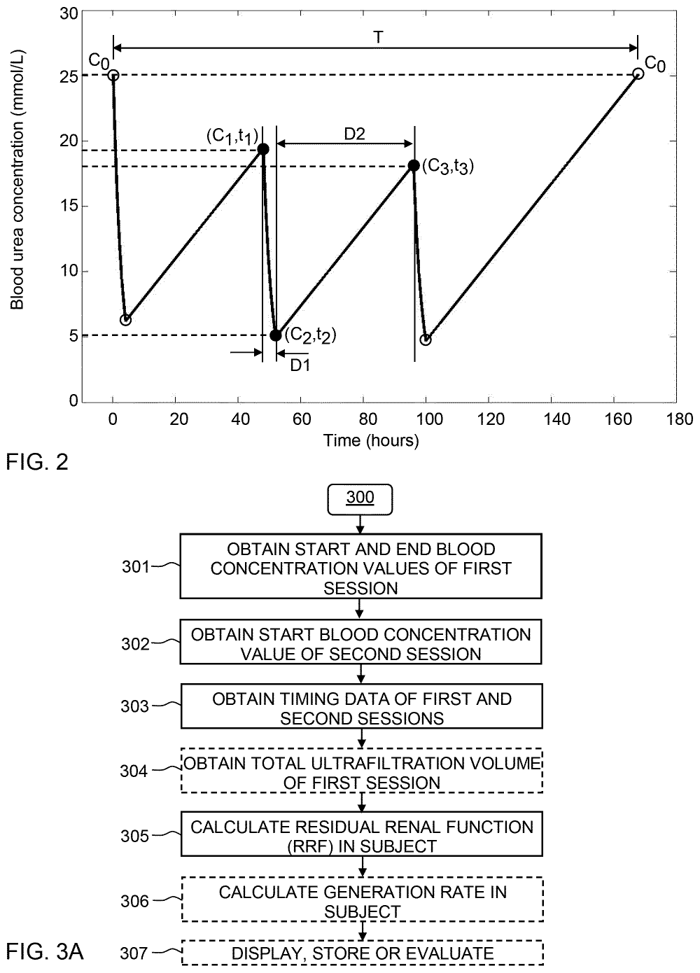

FIG. 2 shows an example of the equilibrated urea concentration in the blood of the patient 100 over a time period of a week, in which the patient is subjected to intermittent dialysis in three separate treatment sessions. In each treatment session, the EC circuit 10 is connected to the vascular system of the patient 100, as shown in FIG. 1, whereupon the system 1 is operated to perform a blood treatment procedure in which uremic solutes and water are removed from the patient's blood via the dialyzer membrane 11a. Such solutes include, without limitation, urea, creatinine, β-2-microglobin (B2M), β-trace protein, cystatin C, Vitamin B12, etc. As seen, the respective treatment session results in a significant reduction in the blood urea concentration. Between the treatment sessions, the blood urea concentration rises as a result of metabolic processes in the patient.

FIG. 2 is characteristic of dialysis therapy performed three times each week, e.g. Monday, Wednesday and Friday. As seen, the equilibrated urea concentrations C0 at the beginning of the week and the end of the week are approximately the same. In fact, all dialysis therapies may be sub-divided into time periods that start and end at approximately the same equilibrated urea concentration. Commonly, the time period is one or more days or one or more weeks. This time period is thus the therapy period T in the definition of stdKt/V. FIG. 2 also illustrates that the pre-dialysis blood urea concentration decreases monotonically from the first to the last session within the therapy period. This is a characteristic of dialysis therapies that involve two or more temporally spaced treatment sessions during the therapy period, in which at least one temporal spacing differs from other temporal spacing(s).

In FIG. 2, the duration D1 of a selected session is indicated, and well as the intermediate time period D2 between the end of the selected session and the start of the next session. During the selected session, the equilibrated urea concentration drops from C1 to C2 between time points t1, t2 and rises from C2 to C3 between time points t2, t3.

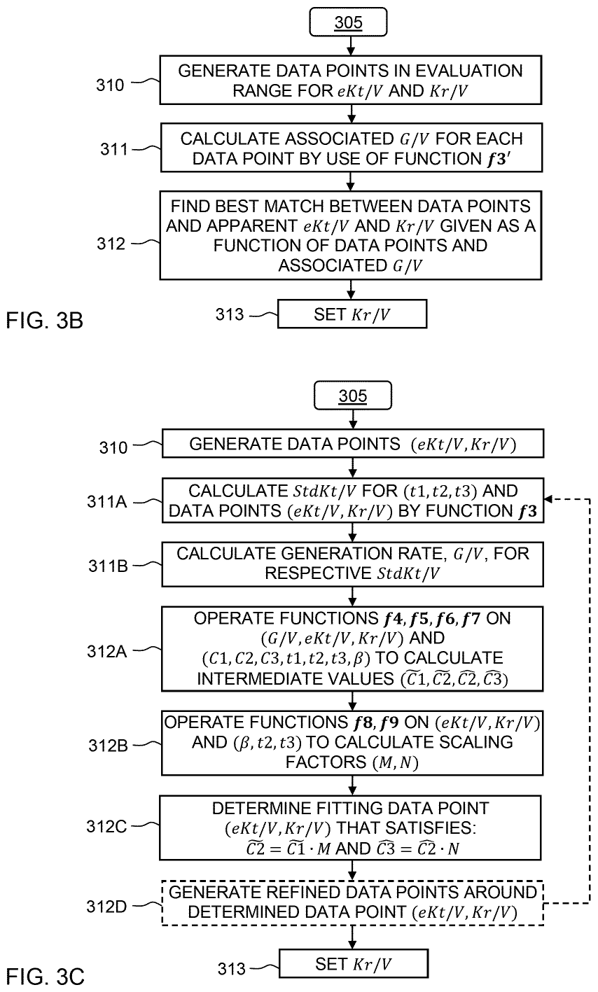

FIG. 3A is a flow chart of an example method 300 for estimating RRF in accordance with an embodiment. The method 300 may be performed by the computer system 40 in FIG. 1 and will be described with reference to FIG. 2. Step 301 obtains concentration values of a substance (e.g. urea in FIG. 2) in the blood of the patient at the start and at the end of a first session (cf. concentration values C1, C2 in FIG. 2). Step 302 obtains a concentration value of the substance in the blood of the patient at the start of a second session that follows upon the first session (cf. concentration value C3 in FIG. 2). In the following examples, it is assumed that all concentration values are equilibrated concentrations since this has been found to significantly improve the accuracy of the method 300. The concentration values may be generated by blood sampling and analysis as described with reference to FIG. 1. Step 303 obtains timing data of the first and second sessions, including at least the time points for the start and end of the first session and the start of the second session (cf. time points t1, t2, t3 in FIG. 2). Steps 301-303 may be performed by manual entry into the computer system 40 or electronic transfer from the blood analysis apparatus 50. Step 304 is optional and obtains the total UF volume (UFV) for the first session. As an alternative to step 304, the method 300 may operate on a default value for the total UF volume or a default value of a corresponding parameter β (see below). Step 304 may be performed by manual entry or by electronic transfer from the control device 30. Step 305 estimates the RRF as a function of measurement data comprising the concentration values from steps 301-302 and the time points from step 303, and optionally the total UF volume from step 304. A motivation for step 305 and various implementation examples will be presented below. The method 300 may further include an optional step 306 that estimates the generation rate of the substance in the patient. Step 307 is likewise optional and may store and/or present the estimated RRF, and optionally the estimated generation rate, for example on the output device 45 in FIG. 1. Alternatively or additionally, step 307 may evaluate the estimated RRF and/or the estimated generation rate for assessment of the physiological status of the patient and may also store and/or present an indicator of the physiological status.

The substance may be any endogenous marker that is generated by the patient, present in the blood, and exchanged with the dialysis fluid during dialysis therapy. Non-limiting examples of such markers include urea, creatinine, beta-2-microglobin, β-trace protein, and cystatin C. In the following examples, it will be assumed that the substance is urea.

Step 305 will now be further explained and motivated with reference to FIG. 2 and by use of equations that are taken or inferred from the above-mentioned article by Sternby. The presented equations are merely given as examples, and the following description is equally appliable to other equations which may be derived to be more approximative or more exact in one or more aspects. It is assumed that the concentration values C1, C2, C3 and the corresponding time points t1, t2, t3 are known (cf. steps 301-303), and that the concentration values represent the equilibrated urea concentration in the blood of the patient.

The session Kt/V for the selected session in FIG. 2 may be calculated according to:

$$eKt / V = - \frac{β}{ln ( 1 + β )} \cdot ln ( \frac{R - α}{1 - α} ) + β \\ α = G / C 1 / ( K + UFr ) \\ β = UFV / V \\ R = C 2 / C 1$$

with eKt/V being the equilibrated session Kt/V, UFV being the total fluid volume removed from the blood during the session, V being the distribution volume in the patient, UFr being the UF rate, K being the equilibrated clearance, and G being the urea generation rate of the patient. The equilibrated clearance K includes the residual clearance Kr. Thus, the effect of RRF is inherently included in the session Kt/V calculated by these equations.

It may be noted that, since UFr·D1=UFV, α may be rewritten as α=G/V·D1/(eKt/V+β)/C1. This yields the following calculation function for eKt/V:

eKt/V=f1(G/V; C1, C2, t1,t2,β) (1)

with G/V being a relative generation rate. It is to be noted that f1 is an implicit function, in which eKt/V is dependent on itself. This means that f1 may be solved iteratively if G/V is known or has been estimated. A solution is likely to be found since f1 is relatively insensitive to changes in eKt/V, which increases the likelihood of convergence.

For the intermediate time period D2 in FIG. 2, the relative residual clearance Kr/V may be calculated according to:

$$Kr / V = 1 / D 2 \cdot ( - \frac{β}{ln ( 1 + β )} \cdot ln ( \frac{{R}^{*} - {α}^{*}}{1 - {α}^{*}} ) + β ) \\ {α}^{*} = G / C 2 / ( Kr - UFr ) \\ {R}^{*} = C 3 / C 2$$

Again, it may be noted that, α* may be rewritten as α^{*}=G/V·D2/(Kr/V·D2−β)/C2. This yields the following calculation function for Kr/V:

Kr/V=f2 (G/V; C2, C3, t2, t3, β) (2)

Here, β is UFV/V for the second session and may or may not be assumed to be the same as for the first session. For simplicity of presentation, β is assumed to be the same for the first and second sessions in the following examples.

Like f1 above, f2 is an implicit function, in which Kr/V is dependent on itself. Theoretically, f2 may be solved iteratively if G/V is known or has been estimated.

As noted above, the definition of stdKt/V is:

stdKt/V=(G·T)/(C̅s̅·V) (3)

with T being the therapy period, and C̅s̅ being the average pre-dialysis concentration during the therapy period. In the example of FIG. 2, T is one week (168 hours).

The article by Sternby defines a general estimation function for stdKt/V that obviates the need to know the generation rate G. The estimation function has an intricate dependence on scale factors N, M (below), durations of treatment sessions, and time periods between treatment sessions. To simplify the present disclosure, it is assumed that all treatment sessions in FIG. 2 have equal duration D1 and that the estimation function is represented by:

stdKt/V=f3 (eKt/V, Kr/V; t1, t2, t3, T) (4)

Combining Eq. 3 and Eq. 4 and rearranging yields the conversion function:

G/V=f3′(eKt/V, Kr/V; t1, t2, t3, T, C̅s̅) (5)

It may be noted that functions f1, f2, f3′ are presented with a semicolon separating variables (unknown data) from parameters (known data). The concentration values C1, C2, C3 and the time points t1, t2, t3 are input by steps 301-303. The parameter β may be given by UFV from step 304 divided by the distribution volume V, which is turn may be predefined or calculated based on the weight of the patient. Alternatively, the parameter β may be set to a predefined value. The therapy time period T may be predefined or entered in a separate step (not shown). The average pre-dialysis concentration C̅s̅ may be calculated from the concentration values C1, C3 or otherwise estimated and/or input. Thus, any and all of the known parameters may be applied to populate the respective function f1, f2, f3′. In this context, the term “populate” infers that the values of the known parameters are entered into the respective function. The unknown variables are limited to eKt/V, Kr/V and G/V. It is seen from Eq. 1 that eKt/V is affected by the variable G/V, from Eq. 2 that Kr/V is affected by the variable G/V, and from Eq. 5 that G/V is affected by the variables eKt/V and Kr/V. It should be understood that the calculation functions f1, f2 (Eq. 1-2) are not sufficient to allow for determination of the unknown variables eKt/V, G/V, Kr/V. The creative definition of the conversion function f3′ (Eq. 5) provides an additional functional relation that makes it possible to determine Kr/V as an estimation value of RRF, as well as eKt/V and G/V if desired.

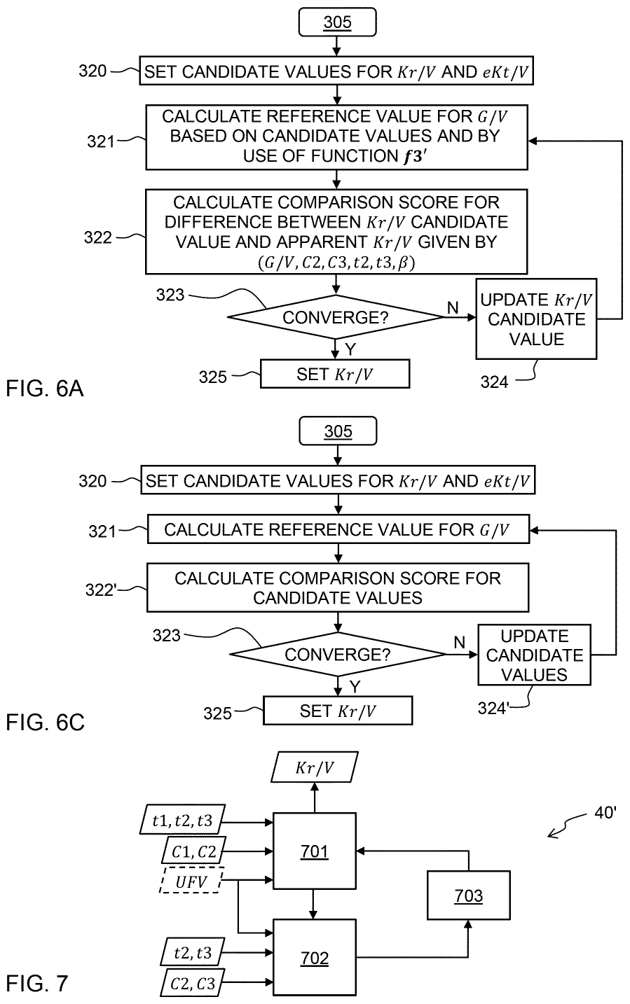

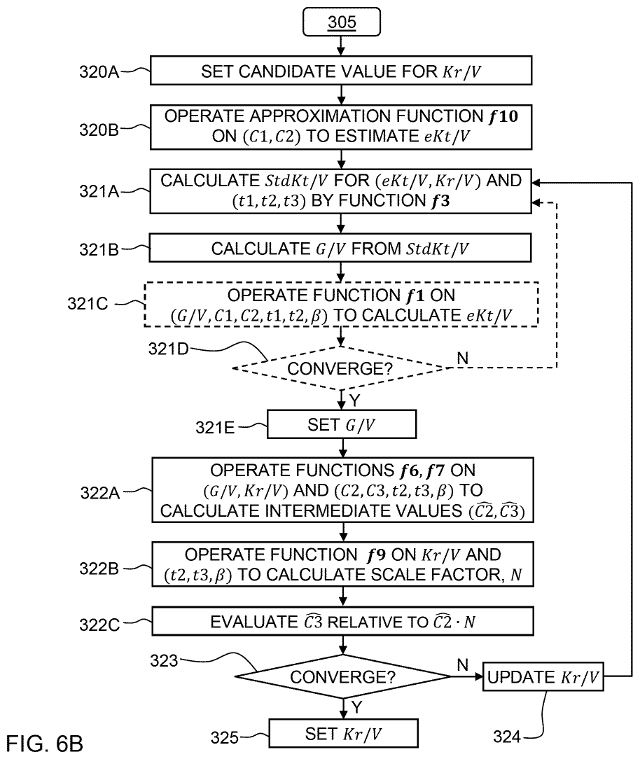

The functions f1, f2, f3′ are highly non-linear with respect to the unknown variables. Thus, the calculations in step 305 need to be carefully implemented to provide a consistent and accurate estimation of the RRF. Below, two concepts of the calculation step 305 will be presented with reference to FIGS. 3B-3C and FIGS. 6A-6C, respectively.

A first approach for performing the calculation step 305 is depicted in the flow chart of FIG. 3B. Step 310 generates a plurality of data points comprising different combinations of values for eKt/V and Kr/V. The data points may be generated to be dispersed within evaluation ranges for eKt/V and Kr/V. The evaluation ranges may be predefined or input by a user. In one example, the evaluation ranges are set to span all possible or reasonable values of eKt/V and Kr/V. In another example, the evaluation ranges may be set in relation to historic data for the patient or similar patients, for example previously estimated values of eKt/V and Kr/V. Step 311 calculates an associated value of G/V for each data point by use of the conversion function f3′ (Eq. 5). Thus, subsequent to step 311, the procedure 305 has generated a plurality of tuples, each containing a unique combination of values: (eKt/V, Kr/V, G/V). Step 312 then processes the tuples to find the best match between the tuples and “apparent values” of eKt/V and Kr/V given as a function of the values of the respective tuple. Step 312 may be seen to determine or find a matching data point among the plurality of data points generated by step 310. Step 313 sets the estimation value of Kr/V to the corresponding value in the matching data point. Similarly, an estimation value of G/V or eKt/V may be set to the corresponding value in the tuple of the matching data point.

In some embodiments, step 312 may directly calculate the apparent values by operating functions f1, f2 on the values in the tuple and related measurement data. In other embodiments, step 312 may indirectly find the best match by use of a re-arrangement of the functions f1, f2 into functional relations between a set of intermediate variables. The re-arrangement may serve to improve the processing-efficiency for finding the best match.

FIG. 3C is a flow chart of a detailed example of the first approach in FIG. 3B, and specifically an example that uses the re-arrangement of the functions f1, f2. The illustrated example includes a step 310 that is identical to step 310 in FIG. 3B, and steps 311A-311B that correspond to step 311 in FIG. 3B. Steps 311A-311B serve to illustrate that the values for G/V in each tuple may be generated by first using the function f3 (Eq. 4) to calculate a value for stdKt/V (step 311A), and then using the definition of stdKt/V (Eq. 3) to calculate the value for G/V based on the value of stdKt/V (step 311B).

Steps 312A-312C in FIG. 3C correspond to step 312 in FIG. 3B and operate on intermediate variables that are presented in the aforesaid article by Sternby, in which they are used for deriving the estimation function for stdKt/V (cf. Eq. 4). However, it has now been realized that these intermediate variables may also be used for finding the best match, and thus Kr/V. Accordingly, the intermediate variables may be given by the following set of equations:

[symbol]

=C1−G/V·a

[symbol]

=C2−G/V·a

[symbol]

=C2−G/V·b

[symbol]

=C3−G/V·b

a=D1/(eKt/V−β)

b=D2/(Kr/V·D2+β)

This may be simplified into the following intermediate functions:

[symbol]

=f4(G/V, eKt/V; C1, t1, t2, β) (6)

[symbol]

=f5 (G/V, eKt/V; C2, t1, t2, β) (7)

[symbol]

=f6(G/V, Kr/V; C2, t2, t3, β) (8)

[symbol]

=f7(G/V, Kr/V; C3, t2, t3, β) (9)

Further, the functional relations between the intermediate variables C1, C2, C2, C3 are given by: [symbol] = [symbol] ·M and [symbol] = [symbol] ·N, with scaling variables M, N being given by:

M=exp(−ln(1+β)/βeKt/V+ln(1+β))

N=exp(−ln(1+β)/βKr/V·D2−ln(1+β))

This may be simplified into the following scaling functions:

M=f8(eKt/V; β) (10)

N=f9(Kr/V; t2, t3, β) (11)

Step 312A operates the intermediate functions f4, f5, f6, f7 on the values (eKt/V, Kr/V, G/V) in the respective tuple, given by steps 310-311, and on related measurement data given by steps 301-303 (FIG. 3A). This measurement data includes C1, C2, C3 and t1, t2, t3. Step 312A also requires a value of β. The result of step 312A are values of the intermediate variables [symbol] , [symbol] , [symbol] , [symbol] for each tuple.

Step 312B operates the scaling functions f8, f9 on the values (eKt/V, Kr/V) in the respective tuple, given by step 310, and on related measurement data given by steps 301-303 (FIG. 3A). This measurement data includes t1, t2. Step 312B also requires the value of β. The result of step 312B are values (“scale factors”) of the scaling variables N, M for each tuple.

Step 312C evaluates the functional relations [symbol] = [symbol] ·M and [symbol] = [symbol] ·N for all data points to determine the data point that provides the best match to both of these functional relations. Thereby, step 312C finds, among the plurality of data points, a fitting data point for which the intermediate value [symbol] substantially equals the intermediate value [symbol] multiplied by the scaling factor M, and for which the intermediate value [symbol] substantially equals the intermediate value [symbol] multiplied by the scale factor N. Step 312C may be performed by generating a first contour curve for M· [symbol] / [symbol] =1 and a second contour curve for N· [symbol] / [symbol] =1 for the data points, i.e. as a function of eKt/V and Kr/V, and determining the data point where the first and second contour curves intersect. The first contour curve is thus a first curve that connects data points where M· [symbol] / [symbol] =1, and the second contour curve is a second curve that connects data points where N· [symbol] / [symbol] =1. Subsequent to step 312C, step 313 may set the estimation value of RRF to the value of Kr/V in this data point.

Step 312C may, for example, be performed by use of a commercially available software program for computational mathematics. In one non-limiting example, MATLAB® provides a predefined function CONTOUR for determining contour plots based on matrices of data.

FIG. 5A shows first and second contour curves 501, 502 that have been generated by the method 300, which includes a step 305 that is implemented according to the procedure in FIG. 3C. The results in FIG. 5A have been generated based on the concentration and timing data shown in FIG. 2, i.e. from the start of the mid-session to the start of the last session in a treatment schedule of three treatment sessions per week (Monday, Wednesday and Friday). The therapy period is set to one week. Each treatment session has a clearance of 250 mL/min and results in a weight loss (UFV) of 2 L. The patient has a distribution volume (V) of 40 L and an actual residual urea clearance (Kr) of 3.5 mL/min. The actual eKt/V for the mid-session is 1.3077. The contour plots 501, 502 in FIG. 5A have been generated for data points in an evaluation range of 1.2-1.5 for eKt/V and an evaluation range of 0-5 mL/min for Kr. As seen, the contour curves 501, 502 meet at an intersection point 503 corresponding to eKt/V=1.31 and Kr=3.5 mL/min, which are both in excellent agreement with the actual values. FIG. 5A also illustrates that the use of Kr/V or Kr as the unknown RRF variable in the embodiments described herein is a matter of choice.

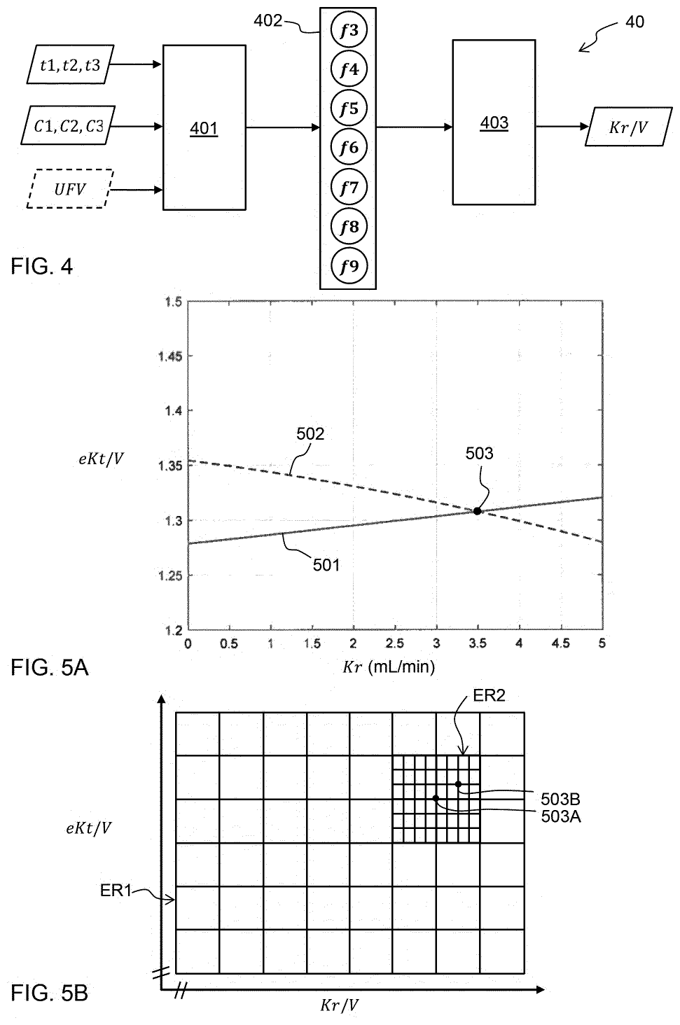

FIG. 4 is a block diagram of an example computer system 40 (cf. FIG. 1) that implements the method 300 as depicted in FIGS. 3A and 3C. The computer system 40 comprises a first module 401 configured to obtain input data, including C1, C2, C3 and t1, t2, t3 and optionally UFV (or β), in accordance with steps 301-304. The input data may also comprise one or more of the therapy period T, the weight of the patient, the distribution volume of the patient, etc. The first module 401 is further configured to generate the data points in accordance with step 310. A second module 402 is configured to receive the data points and the input data from the first module 401 and evaluate the functions f3-f8 based thereon, in accordance with steps 311A, 311B, 312A and 312B. The resulting intermediate values [symbol] , [symbol] , [symbol] , [symbol] and the scaling factors N, M for each data point are received by a third module 403, which determines the data point that provides the best match to the two functional relations ( [symbol] = [symbol] ·M and [symbol] = [symbol] ·N), in accordance with step 312C. The third module 403 then generates the estimation value of the RRF based thereon, for example in the form of Kr or Kr/V.

Reverting to FIG. 3C, it is seen that the procedure 305 may comprise an optional refinement step 312D, which generates a refined plurality of data points based on the data point that has been identified as the best match by step 312C, whereupon steps 311A-312C are repeated for the refined data points. The operation of step 312D is illustrated graphically in FIG. 5B. Here, step 310 has defined a first grid ER1 of data points and step 312C has determined, for the first grid ER1, that the data point 503A is the best match. Then, step 312D may generate a second grid ER2, which has a higher resolution and a smaller extent than the first grid ER1 and is centered on or otherwise arranged around the data point 503A. Here, a “higher resolution” infers that the distance between neighboring data points is decreased for at least one of the variables that define the data points, i.e. along at least one eKt/V or Kr/V in the example of FIG. 5B. By performing steps 311A-312C for the second grid ER2 of data points, it is possible to increase the accuracy of the best match determined by step 312C, as exemplified by data point 503B in the second grid ER2. The inclusion of step 312D provides a processing-efficient way of increasing the accuracy of the procedure 305 in FIG. 3C.

A second approach for performing the calculation step 305 is depicted in the flow chart of FIG. 6A. Step 320 sets candidate values for Kr/V and eKt/V (“first and second candidate values”). In step 320, the candidate value of eKt/V may be estimated by use of an approximation function (cf. f10 in step 320B in FIG. 6B). The candidate values form starting values for subsequent iterations through steps 321-324. Step 321 calculates a reference value for G/V (“G/V reference value”) based on the candidate values and by use of the conversion function f3′ (Eq. 5). Step 321 may also calculate a refined value of the eKt/V candidate value as part of calculating the G/V reference value (cf. steps 321A-321E in FIG. 6B). Step 322 then calculates a comparison score which is indicative of the relation between the Kr/V candidate value and an apparent value of Kr/V (“apparent RRF”), which is given by the G/V reference value, the concentration values C1, C2, the time points t1, t2, as well as β. Step 322 evaluates the comparison score in relation to a convergence criterion, which may be defined to indicate a sufficient similarity between the Kr/V candidate value and the apparent RRF. If the convergence criterion is not fulfilled, the procedure proceeds to step 324, which updates the Kr/V candidate value and then proceeds to step 321. If the convergence criterion is fulfilled, step 325 may set the estimation value of the RRF to the Kr/V candidate value or the apparent RRF (if calculated, see below). The updating by step 324 may be performed by use of any known technique for steering an iterative procedure towards a known target value. For example, such known techniques include first order methods such as the bisection method or the Regula-Falsi method, and second order methods such as the Newton-Raphson method.

In some embodiments, step 322 may directly calculate the apparent RRF by evaluating the function f2, and calculate the comparison score based thereon. In one non-limiting example, the comparison score may be given by: (Kr/V−f2( . . . ))², where Kr/V represents the Kr/V candidate value and f2( . . . ) represents the apparent RRF.

In other embodiments, step 322 may indirectly determine the comparison score by use of the re-arrangement of the function f2 into a functional relation between a set of intermediate variables, as described above. The re-arrangement may serve to improve the stability of the procedure 305 by reducing the risk that the iterations through steps 321-324 do not converge.

FIG. 6B is a flow chart of a detailed example of the second approach in FIG. 6A, and specifically an example that uses the re-arrangement of the function f2. The illustrated example includes steps 320A-320B that correspond to step 320 in FIG. 6A. Step 320A sets a Kr/V candidate value. Step 320B operates an approximation function f10 on the available measurement data to estimate eKt/V for the selected treatment session. In the example of FIG. 2, the function f10 may operate on C1, C2, UFV and the weight (W) of the patient at the end of the selected session. The approximation function f10 may, for example, be the established Daugirdas formula for estimating single-pool Kt/V (spKt/V), which represents eKt/V if operated on equilibrated concentrations:

eKt/V=−ln(R−0.008·(t2−t1))+(4−3.5·R)·UFV/W

The function f10 is not limited to this example but may be any known function for approximating eKt/V based on the available measurement data.

Steps 321A-321B correspond to step 321 in FIG. 6A. Step 321A calculates stdKt/V for the Kr/V and eKt/V candidate values given by steps 320A-320B, by use of the function f3 (Eq. 4) and based on the time points t1, t2, t3. Step 321B then calculates G/V by use of the definition of stdKt/V (Eq. 3). It is realized that steps 321A and 321B may be merged to calculate G/V by use of the conversion function f3′ (Eq. 5). The resulting G/V may be set by step 321E for use by subsequent steps.

The G/V calculated by step 321B is a rather coarse approximation as a result of the use of the approximation function f10 in step 320B. The accuracy of G/V may be improved by an iterative procedure, represented by optional steps 321C-321D in FIG. 6B. Step 321C operates calculation function f1 on G/V given by step 321B, to calculate a refined value of eKt/V. Step 321D evaluates the refined value in relation to a convergence criterion, which may be defined to indicate a sufficient similarity between eKt/V values and/or G/V values between iterations. If the convergence criterion is not fulfilled, the procedure proceeds to step 321A, which performs the calculation of stdKt/V for the refined value of eKt/V. Step 321D may thus be seen to provide an updated value of the eKt/V candidate value to be used in the next iteration of steps 321A-321D. If the convergence criterion is fulfilled, step 321E sets G/V to the latest value calculated by step 321B.

Subsequent to step 321E, the procedure 305 proceeds to steps 322A-322C, which correspond to the calculation of the comparison score by step 322 in FIG. 6A. Instead of calculating the comparison score for an explicit apparent RRF given by function f2, steps 322A-322C calculates the comparison score based on the intermediate and scaling functions that correspond to function f2. Step 322A operates intermediate functions f6 and f7 on G/V (from step 321E) and the Kr/V candidate value, as well as related measurement data, to calculate the intermediate values [symbol] , [symbol] . Step 322B operates scaling function f9 on the Kr/V candidate value and related measurement data to calculate the scale factor N. Step 322C evaluates the relation between [symbol] and [symbol] ·N, for example by calculating [symbol] /( [symbol] ·N). Recalling the functional relation [symbol] = [symbol] ·N, it is realized that the evaluation of step 322C corresponds to calculating the comparison score in step 322. Step 323 evaluates the output of step 322C in relation to a convergence criterion, which may be defined to indicate a sufficient similarity between [symbol] and [symbol] ·N. If the convergence criterion is not fulfilled, step 324 updates the candidate value and then proceeds to step 321A. If the convergence criterion is fulfilled, step 325 may set the estimation value of Kr/V to the latest value from step 324. As noted above, step 324 may be performed by use of any known technique for steering, for example, [symbol] /( [symbol] ·N) towards a value of 1.

FIG. 6C is a flow chart of a variant of the second approach, which differs from the example in FIG. 6A by steps 322′ and 324′. Step 322′ calculates the comparison score to be indicative of the relation between the candidate values and corresponding apparent values of eKt/V and Kr/V. The apparent value of eKt/V may be given by calculation function f1, and the apparent value of Kr/V may be given by calculation function f2. In one non-limiting example, the comparison score may be given by: (eKt/V−f1( . . . ))²+w·(Kr/V−f2( . . . ))², where eKt/V, Kr/V represent the candidate values, f1( . . . ), f2( . . . ) represent the apparent values, and w is an optional weight which may be set to make the first and second terms of comparable magnitude. Step 323 evaluates the comparison score in relation to a convergence criterion, which may be defined to indicate a sufficient similarity between the candidate values and the apparent values. If the convergence criterion is not fulfilled, the procedure proceeds to step 324′, which updates both of the candidate values and then proceeds to step 321. If the convergence criterion is fulfilled, step 325 may set the estimation value of the RRF to the Kr/V candidate value or the apparent value of Kr/V.

FIG. 7 is a block diagram of an example module 40′ of the computer system 40 (cf. FIG. 1) that implements the procedure 305 as depicted in FIGS. 6A-6C. The module 40′ comprises a first sub-module 701 configured to obtain input data, including C1, C2 and t1, t2, t3, and optionally UFV (or β), and perform steps 320-321 (FIGS. 6A, 6C) or steps 320A-321E (FIG. 6B). The sub-module 701 is thereby configured to generate and output a value of G/V. A second sub-module 702 is configured to obtain input data, including C2, C3, t2, t3, and optionally UFV (or β), and perform step 322 (FIG. 6A), steps 322A-322C (FIG. 6B) or step 322′ (FIG. 6C). The sub-module 702 is thereby configured to output the comparison score. In the context of FIGS. 6A-6B, the third sub-module 703 is configured to receive the comparison score, perform steps 323-324, and output an updated Kr/V candidate value for use by sub-module 701. In the context of FIG. 6C, the third sub-module 703 is configured to receive the comparison score, perform steps 323-324′, and output updated Kr/V and eKt/V candidate values for use by sub-module 701. The sub-module 703 is further configured to signal to sub-module 701 when convergence is detected, causing sub-module 701 to output the estimation value of RRF, for example Kr/V.

Reverting to Eq. 3 and Eq. 5, these equations require the average pre-dialysis concentration C̅s̅ during the therapy period T to be known. In the example of FIG. 2, the true value of C̅s̅ is equal to the average of C0, C1, C2. Since each concentration value may require blood sampling and analysis, it may be desirable to estimate C̅s̅ based on fewer concentration values, for example the concentration values obtained by steps 301-302. From FIG. 2, it is realized that among the pre-dialysis concentration values C0, C1, C2, the value C1 lies closest to the true C̅s̅. Thus, the estimated value of C̅s̅ may be given by C1 or set in relation to C1, e.g. by multiplication with a predetermined correction factor and/or addition of a predetermined correction value (positive or negative). It may also be seen in FIG. 2, that it may be beneficial to perform the method 300 for the first or last treatment session during the therapy period T, since the average of the available pre-dialysis concentrations (C0, C1 for the first session, and C3, C0 for the last session) is a fair approximation of the true C̅s̅. Generally, the estimated value of C̅s̅ may be given by a predetermined function of one or several concentration values.

As noted above, the distribution volume V of the patient may be used by the method 300 in some embodiments. The distribution volume may be estimated in various ways known to the skilled person. For example, the distribution volume V may be approximated by the total body water (TBW), which may be estimated for the patient. For example, the caretaker may input the dry weight or body weight of the patient, and possibly further patient data such as sex, age, height, etc, thereby allowing the method 300 to estimate TBW of the patient 100, e.g. by assuming that TBW is a given percentage of the body weight of the patient, or by using any established formula such as the Watson formula, the Hume-Weyers formula or the Chertow formula. Alternatively, TBW may be measured on the patient, e.g. by bioelectrical impedance analysis (BIA) or a dilution method.

While the subject of the present disclosure has been described in connection with what is presently considered to be the most practical and preferred embodiments, it is to be understood that the subject of the present disclosure is not to be limited to the disclosed embodiments, but on the contrary, is intended to cover various modifications and equivalent arrangements included within the spirit and the scope of the appended claims.

Further, while operations are depicted in the drawings in a particular order, this should not be understood as requiring that such operations be performed in the particular order shown or in sequential order, or that all illustrated operations be performed, to achieve desirable results. In certain circumstances, parallel processing may be advantageous.

In the foregoing examples, the conversion function f3 was derived under the assumption that all treatment sessions within the therapy period T have equal duration. However, a corresponding function, albeit more complicated, may be derived even if some or all treatment sessions have different durations and/or if the time periods between the treatment sessions differ, for example based on the equations provided in the above-mentioned articles. Such implementations may require additional measurement data such as concentration values and time points for all treatment sessions within the therapy period. Further, as noted above, the calculations may need to be adapted if UFV differs between sessions, resulting in differences in β between sessions. Such adaptations should be readily apparent to the skilled person.

Further, the example method 300 may be performed for more than one pair of consecutive treatment sessions, during the same or different therapy periods T. In such embodiments, one estimation value of RRF may be calculated for each pair of consecutive treatment sessions, in accordance with the method 300, and a final estimation value may be given by the average of the calculated estimation values.

Correspondingly, the example method 300 may be performed for more than one substance, for example urea and creatinine, and a final estimation value may be given by a (weighted) average of the calculated estimation values.

In the example of FIG. 1, the concentration values are given by blood sample analysis, and possibly subsequent conversion into equilibrated concentrations. In an alternative, the blood treatment system 1 comprises a device that calculates the in-vivo clearance of the respective treatment session, for example based on conductivity measurements in the dialysis fluid and effluent paths 13a, 14a. Examples of commercially available measuring devices include DIASCAN from Gambro/Baxter, and Online Clearance Monitoring (OCM) from Fresenius. Depending on implementation, such a measuring device may output a clearance value K or a corresponding session Kt/V for the respective treatment session. It is conceivable to process the clearance value K or the session Kt/V to generate corresponding (equilibrated) concentration values at the start and end of a treatment session. Thus, in some embodiments, the computer system 40 may be configured to receive output data from such a measuring device and calculate the required blood concentration values based at least partly thereon.

1. A computer-implemented method of estimating a residual renal function (“RRF”) in a dialysis patient, said method comprising:
obtaining first and second concentration values (C1, C2) for a substance in blood of the dialysis patient at a start and an end of a first treatment session of intermittent dialysis therapy, and a third concentration value (C3) for a substance in the blood of the dialysis patient at a start of a second treatment session, which is consecutive to the first treatment session, the substance being selected from the group of urea, creatinine, beta-2-microglobin, β-trace protein, and cystatin C;
obtaining time points (t1, t2, t3) for the start and the end of the first treatment session and the start of the second treatment session; and
calculating an estimation value (Kr/V) of the RRF as a function of the first, second, and third concentration values (C1, C2, C3), and the time points (t1,t2,t3),
wherein the first and second treatment sessions are performed during a therapy time period T,
wherein calculating the estimation value (Kt/V) is based on an assumption that the concentration of the substance in the blood of the dialysis patient is substantially equal at a start and an end of the therapy time period (T), and
wherein calculating the estimation value (Kt/V) comprises:
representing an unknown generation rate (G/V) of the substance in the dialysis patient by a functional dependence on a standard Kt/V for the therapy time period (T),
representing the standard Kt/V by a predefined estimation function (f3), which is based on the assumption and operates on the time points (t1,t2,t3), the therapy time period (T), an unknown session Kt/V of the first treatment session, and the RRF to be estimated.
representing the unknown session Kt/V of the first treatment session by a first calculation function (f1), which operates on the unknown generation rate (G/V), the first and second concentration values (C1, C2), and the time points (t1, t2) for the start and the end of the first treatment session, and
representing the estimation value (Kt/V) by a second calculation function (f2), which operates on the unknown generation rate (G/V), the second and third concentration values (C2, C3), and the time points (t2, t3) for the end of the first treatment session and the start of the second treatment session.

2. The method of claim 1, wherein the estimation value (Kt/V) of the RRF is calculated independently of data representing urine collected from the dialysis patient.

3. The method of claim 1, wherein the first, seconds and third concentration values (C1, C2, C3) represent equilibrated concentrations of the substance in the blood of the dialysis patient.

4. (canceled).

5. The method of claim 1, wherein the therapy time period (T) comprises a week.

6. (canceled).

7. The method of claim 1, wherein said functional dependence comprises multiplying the standard Kt/V, and an estimated concentration value (C̅s̅), which is representative of an average pre-dialysis concentration of the substance in the blood of the dialysis patient during the therapy time period (T), and the reciprocal of the therapy time period (T).

8. The method of claim 1, wherein said calculating the estimation value (Kr/V) comprises:
generating a plurality of data points, each of the data points comprising a unique combination of candidate values of the session Kt/V and the RRF;
calculating, for a respective data point, an associated value of the generation rate (G/V) by use of the predefined estimation function (f3) populated by the time points (t1, t2, t3);
determining a matching data point among the plurality of data points; and
setting the estimation value (Kr/V) to the candidate value of the RRF in the matching data point,
wherein the candidate values of the matching data point match an apparent session Kt/V for the first treatment session and an apparent RRF,
wherein the apparent session Kt/V is a function of the first and second concentration values (C1, C2), the time points (t1, t2) for the start and the end of the first treatment session, and the associated value of the generation rate (G/V) for the matching data point, and
wherein the apparent RRF is a function of the second and third concentration values (C2, C3), the time points (t2, t3) for the end of the first treatment session and the start of the second treatment session, the associated value of the generation rate (G/V) for the matching data point.

9. The method of claim 8, wherein determining the matching data point among the plurality of data points comprises:
operating intermediate functions (f4, f5, f6, f7) on the respective data point and the associated value of the generation rate (G/V), to calculate first, second, third, and fourth intermediate values ( [symbol] , [symbol] , [symbol] , [symbol] );
operating scaling functions (f8, f9) on the respective data point to calculate first and second scale factors (M, N); and
determining the estimation value (Kr/V) as a function of the plurality of data points, the first, second, third, and fourth intermediate values ( [symbol] , [symbol] , [symbol] , [symbol] ) generated for the respective data point, and the first and second scale factors (M, N) generated for the respective data point.

10-11. (canceled).

12. The method of claim 9, wherein determining the estimation value (Kr/V) comprises finding, among the plurality of data points, a fitting data point for which the second intermediate value ( [symbol] ) substantially equals the first intermediate value ( [symbol] ) multiplied by the first scaling factor (M), and for which the fourth intermediate value ( [symbol] ) substantially equals the third intermediate value ( [symbol] ) multiplied by the second scale factor (N),
wherein the matching data point is determined based on the fitting data point.

13. (canceled).

14. The method of claim 1, wherein calculating the estimation value (Kr/V) comprises:
setting a first candidate value of the RRF and a second candidate value of the session Kt/V;
calculating a reference value of the generation rate (G/V) by use of the predefined estimation function (f3) populated by the time points (t1, t2, t3), the therapy time period (T), and the first and second candidate values;
calculating a comparison score indicative of the first candidate value in relation to an apparent RRF given as a function of the reference value, the second and third values (C2, C3), and the time points (t2, t3) for the end of the first treatment session and the start of the second treatment session;
iteratively modifying the first candidate value while calculating the reference value and the comparison score until the comparison score fulfils a convergence criterion; and
setting the estimation value (Kr/V) to the candidate value for which the comparison score fulfils the convergence criterion.

15. (canceled).

16. The method of claim 14, wherein calculating the reference value comprises:
calculating the standard Kt/V by operating the predefined estimation function (f3) on the time points (t1, t2, t3), the therapy time period (T), and the first and second candidate values;
calculating an estimated generation rate (G/V) based on the standard Kt/V; and
determining the reference value based on the estimated generation rate (G/V).

17. The method of claim 16, wherein calculating the reference value further comprises:
updating the second candidate value by operating a refinement function (f1) on the estimated generation rate (G/V), the first and second concentration values (C1, C2), and the time points (t1, t2) for the start and end of the first treatment session; and
repeatedly calculating the standard Kt/V, calculating the estimated generation rate (G/V), and updating the second candidate value, until a second convergence criterion is fulfilled,
wherein determining the reference value based on the estimated generation rate sets the reference value to the estimated generation rate (G/V) for which the second convergence criterion is fulfilled.

18. The method of claim 14, wherein calculating the comparison score comprises:
operating the intermediate functions (f6, f7) on the reference value and the first candidate value to calculate the first and second intermediate values ( [symbol] , [symbol] ); and
operating a scaling function (f9) on the first candidate value to calculate a scale factor (N),
wherein the comparison score defines a relation between the second intermediate value ( [symbol] ) to the product of the first intermediate value ( [symbol] ) and the scale factor (N).

19. The method of claim 18, wherein the intermediate functions (f6, f7) are populated by the second and third concentration values (C2, C3) and the time points (t2, t3) for the end of the first treatment session and the start of the second treatment session, and
wherein the scaling function (f9) is populated by the time points (t2, t3) for the end of the first treatment session and the start of the second treatment session.

20. The method of claim 14, wherein the comparison score is calculated to be further indicative of the second candidate value in relation to an apparent session Kt/V given as a function of the reference value, the first and second values (C1, C2), and the time points (t1, t2) for the start and the end of the first treatment session, and
wherein the first and second candidate values are iteratively modified until the comparison score fulfils the convergence criterion.

21. The method of claim 1, further comprising one or more of: displaying the estimation value (Kr/V), evaluating the estimation value (Kr/V) for assessment of a physiological status of the dialysis patient, and displaying an indicator of the physiological status of the dialysis patient.

22. The method of claim 7, further comprising: calculating the estimated concentration value (C̅s̅) as a function of at least one of the first and third concentration values (C1, C3).

23. The method of claim 22, further comprising: calculating the estimated concentration value (C̅s̅) as a weighted average of at least the first and third concentration values (C1, C3).

24. (canceled).

25. The method of claim 1, further comprising obtaining a volume value (UFV) representative of total fluid volume removed from the blood during the first treatment session,
wherein the estimation value (Kr/V) of the RRF is further calculated as a function of volume value (UFV).

26. A computer-readable medium comprising program instructions which, when executed by a processor, cause the processor to perform the method in accordance with claim 1.

27. A computer system for estimating a residual renal function (“RRF”) in a dialysis patient, said computer system being configured to perform the method in accordance with claim 1.
